(19) United States
(12) Patent Application Publication
(10) Pub. No.: US 2022/0408949 A1
(43) Pub. Date: Dec. 29, 2022

(54) BEVERAGE BOTTLE WITH INTEGRATED AEROSOL SUPPLEMENT

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/839,221

(22) Filed: Jun. 13, 2022

Related U.S. Application Data
(60) Provisional application No. 63/209,826, filed on Jun. 11, 2021.

(51) Int. Cl. A47G 19/22; B65D 47/24; B65D 23/12; B65D 51/28; A47G 23/16; A61M 11/02

(52) U.S. Cl. CPC A47G 19/2272; B65D 47/247; B65D 23/12; B65D 51/28; A47G 23/16; A61M 11/02; A61M 2202/0482; A61M 2210/0625

(57) ABSTRACT

A water bottle with a pressurized compartment and an unpressurized compartment to integrate supplemental oxygen delivery into a hydration and electrolyte management system. The unpressurized compartment may be manually filled with a fluid. The user may consume fluid from the unpressurized compartment and an aerosol from the pressurized compartment independently or simultaneously.

The present application claims to priority to U.S. Provisional Patent Application No. 63/209,826 filed Jun. 11, 2021, which is incorporated by reference.

### FIELD OF THE INVENTION

The present invention is generally related to the delivery nutritional supplements through the oral mucosa, specifically the storage and delivery devices needed to deliver them.

### BACKGROUND

Proper hydration in athletes is essential for optimal performance, and hydration management has long been a laborious task for coaches and trainers. To that end, systems have been developed to monitor hydration in real-time by using water bottles capable of monitoring an athlete's fluid intake. This information can be collected and compared with changes in the athlete's weight to train staff to evaluate an athlete's hydration.

During exercise, the body requires more oxygen than when the body is at rest. When oxygen cannot be delivered to the muscles faster to replace the used oxygen, the muscles will begin to convert the available glucose to lactic acid. This will build up in the muscle, causing fatigue. More rapid delivery of oxygen to the system during exertion can reduce fatigue. Supplemental oxygen therapy has been demonstrated to improve athletic performance and decrease reaction times.

The benefits of proper hydration and supplemental oxygen therapy on athletic performance are demonstrable. Therefore, it is desirable to have a means of integrating the delivery of electrolytes and supplemental oxygen to a real-time hydration management system.

### BRIEF DESCRIPTIONS OF THE DRAWINGS

FIG. 1 illustrates a beverage bottle with an integrated supplement canister.

FIG. 2A illustrates a beverage bottle with an inline supplement canister.

FIG. 2B illustrates the beverage bottle with the inline supplement canister.

FIG. 3 illustrates an external supplement canister for a beverage bottle.

### SUMMARY

A beverage bottle that dispenses both a beverage, such as water, and an aerosol supplement composition is described. The aerosol supplement composition may include electrolytes and other nutritional supplements. The beverage bottle may have separate outlets for the beverage and the aerosol. The beverage bottle may contain a canister of the supplement composition under pressure. The canister may be pressurized with air or oxygen.

In one aspect, a beverage bottle with a pressurized compartment and an unpressurized compartment to integrate supplemental oxygen delivery into a hydration and electrolyte management system is described.

In one aspect, a beverage bottle system to dispense both a fluid and an aerosol supplement composition is described. The system includes a bottle configured to hold a fluid. The bottle defines an interior. A cap is configured to engage the bottle. The cap includes a dispensing opening. A canister contains a supplement composition under pressure. A cradle is configured to support the canister in the system. An outlet connects to the canister. The outlet includes an exterior opening. The exterior opening is configured to dispense an aerosol of the supplement composition. The dispensing opening is configured to dispense the fluid.

In another aspect, a beverage bottle system to dispense both a fluid and an aerosol supplement composition is described. The bottle is configured to hold a fluid. The bottle defines an interior. A cap is configured to engage over the interior of the bottle. The cap includes a dispensing opening. The dispensing opening is configured to dispense the fluid. A canister contains a supplement composition under pressure. An outlet connects to the canister. The outlet includes an exterior opening. The exterior opening is configured to dispense an aerosol of the supplement composition separate from the fluid.

In another aspect, an external supplement canister for a beverage bottle is described. The canister mounts to an exterior of a beverage bottle. An outlet connects to the canister. The outlet includes an exterior opening. The exterior opening is configured to dispense an aerosol of the supplement composition.

In another aspect, an external supplement canister for a beverage bottle is described. The canister includes a mounting bracket configured to extend around an outer circumference of a beverage bottle. The canister contains a supplement composition under pressure. The mounting bracket is configured to hold the canister. An outlet connects to the canister. The outlet includes an exterior opening. The exterior opening is configured to dispense an aerosol of the supplement composition.

### DETAILED DESCRIPTION

For purposes of this application, any terms that describe relative position (e.g., “upper”, “middle”, “lower”, “outer”, “inner”, “above”, “below”, “bottom”, “top”, etc.) refer to an aspect of the invention as illustrated, but those terms do not limit the orientation in which the aspect can be used.

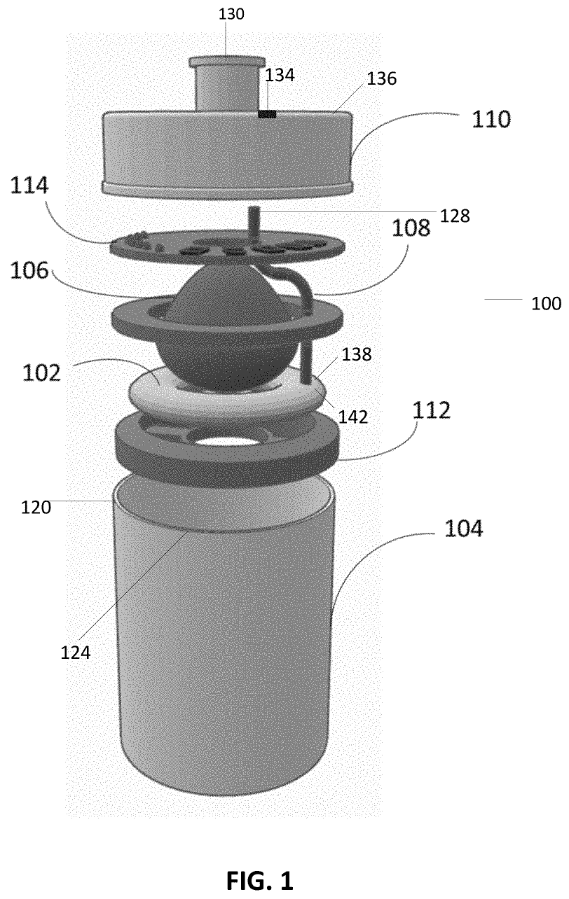

FIG. 1 depicts a system for a beverage bottle with an integrated aerosol supplement 100 to dispense both a fluid and an aerosolized nutritional supplement. The system 100 includes both a pressurized compartment holding a nutritional supplement composition that is dispensed in an aerosol form and an unpressurized compartment configured to hold the fluid. The system 100 may dispense the fluid and the aerosol together or independently. The system 100 may dispense the fluid and the aerosol together or independently from different outlets or dispensers of the beverage bottle.

The system 100 comprises a canister 102 holding a nutritional supplement composition or matrix to enable transmucosal delivery of the nutritional supplement. Transmucosal delivery of nutrient supplements offers advantages over oral delivery when negative issues relating to the gastrointestinal tract, stomach, substance digestion, absorption, swallowing, protocol compliance, substance effectiveness, and other gastrointestinal metabolism issues are considered. The nutritional supplement composition may include (i) a nutritional supplement matrix fraction, (ii) a gas fraction, (iii) an enhancer fraction, (iv) a liquid fraction, and (v) a preservative fraction wherein the nutritional supplement matrix fraction, the gas fraction, the enhancer fraction, the liquid fraction, and the preservation fraction are all mixed or combined and treated to maintain a state of balanced suspension among the oxygen molecules for a specific duration of time before being dispensed from a canister 102. Furthermore, the supplement composition may be in a compressed state in the canister 102 before being dispensed.

With respect to FIG. 1, the bottle 104 includes an opening 120 leading to an interior 124. The bottle 104 may hold fluids, such as water, electrolytic solutions, or other beverages in the interior 124. The cap 110 comprises a dispensing opening 130. The dispensing opening 130 is configured to dispense the fluid and, in certain aspects, the composition. The bottle 104 may include beverage containers of all shapes and sizes. The cap 110 is configured to be removable from the bottle 104 in order to fill the bottle 104 with fluid.

The canister 102 holds the supplement composition in a compressed or pressurized state until the supplement composition is dispensed from the system 100. The canister 102 may be fixed to or positioned in a bottle 104. With respect to FIG. 1, the system 100 includes the canister 102 having a torus-shape. In other aspects, the canister 102 may be provided in different shapes and configurations.

The supplement composition may be delivered to the user via an outlet 108. The outlet 108 may include tubing, hosing, ducts, or piping fluidly connecting the canister 102 and an exterior of the system 100. In certain aspects, the outlet 108 may be configured to direct the supplement composition towards the buccal mucosa. An exterior opening 128 of the outlet 108 may be integrated into the cap 110 or the dispensing opening 130. A lower opening 138 of the outlet 108 may connect with the canister 102.

In the aspect of FIG. 1, the outlet 108 passes through or ends at an opening 134 in an upper surface 136 of the cap 110, and the exterior opening 128 is at or near the opening 134 in the upper surface 136 of the cap 110. The opening 134 is adjacent to the dispensing opening 130. Thus, is this aspect, the dispensing opening 130 provides the fluid and the exterior opening 128 provides the supplement composition, and the exterior opening 128 of the outlet 108 are positioned adjacent to the dispensing opening 130 of the cap 110. In other aspects, the exterior opening 128 of the outlet 108 is positioned within the dispensing opening 130 of the cap 110.

In the aspect of FIG. 1, the cap 110 rotates with respect to the bottle 104 between locked and unlocked configurations. In the unlocked configuration, the user may press downward on the cap 110 towards the interior 124 of the bottle 104 in order to dispense to the supplement composition from the canister 102. Upon actuation by the downward movement of the cap 110, an aerosol of the supplement composition travels from the canister 102, through the outlet 108, and to the exterior opening 128 to dispense from the system 100. The downward movement of the cap 110 may open a valve 142 positioned in the outlet 108 or the canister 102 to allow the supplement composition to dispense. In the locked configuration, the cap 110 is general unable to move downward with respect to the bottle 104. The valve 142 may include a pressure relief valve. In this aspect, the valve 142 is positioned at a junction of the outlet 108 and the canister 102.

In the aspect of FIG. 1, the cradle 112 supports the canister 102. The cradle 112 includes one or more openings to provide for fluid passage from the bottle 104 to the dispensing opening 130 of the cap 110. In this aspect, the cradle 112 is sized and shaped to rest on a rim of the bottle 104.

In the aspect of FIG. 1, a support ring 140 is positioned between the canister 102 and the cap 110. The outlet 108 may pass through an opening in the support ring 140. The support ring 140 may position or stabilize the outlet 108.

In other aspects, the bottle 104 may include a flow measurement device, such as a thermal mass flow meter, to measure the user's amount of fluid consumed.

In other aspects, the user may press directly against the exterior opening 128 of the outlet 108 to dispense the supplement composition from the system 100. In other aspects, the bottle 104 or the cap 110 may include buttons, triggers, etc. to dispense the supplement composition from the system 100.

In other aspects, the system 100 may include a pod 106, which may contain a hydration supplement that is mixed with the fluid in the bottle 104 and delivered to the user through the cap 110. With respect to FIG. 1, the system 100 includes the canister 102 having the torus-shape. In this aspect, the canister 10 with the torus-shape is positioned around pod 106. In other aspects, the cradle 112 may fracture the pod 106 to mix its contents with the fluid in the bottle 104. In other aspects, the cradle 112 may have a flow measurement device to measure the user's amount of fluid.

In certain aspects, the system 100 may include a circular disk 114, which may contain electronics, including a programmable logic controller, a memory to store flow measurements, a transceiver to transmit the measurements, and a battery. In one aspect, the circular disk 114 may contain LED lights to convey usage statistics, schedules, or suggestions to the user, trainer, or coach. In one aspect, the circular disk 114 may contain, or communicate to a computer, a watch, a transdermal patch, or other wearable technology that may analyze the data related to the fluid and nutritional supplement matrix consumption to calculate whether the athlete should consume more or less of either the fluid or the nutritional supplement matrix. In another aspect, the system 100 may be designed to adapt to any water bottle wherein the internal components would be designed at different dimensions to fit different size water bottles. In this aspect, the circular disk 114 is mounted above the cradle 112.

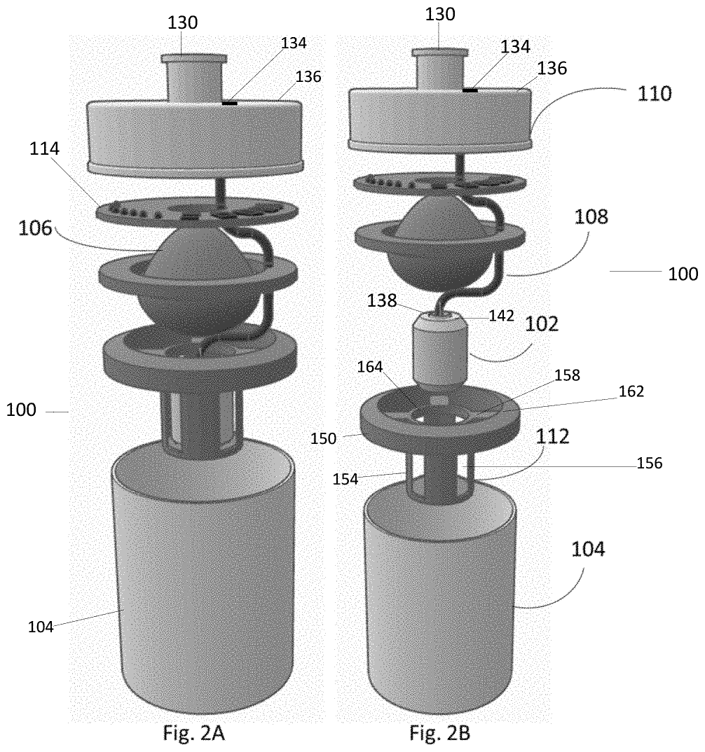

FIGS. 2A and 2B illustrates an aspect of the system 100 with an inline supplement canister 102. In this aspect, the cradle 112 is shaped or configured to accept the nutritional supplement canister 102 inside of the bottle 104. In this aspect, the cradle 112 includes an upper portion 150 and a lower portion 154. The lower portion 154 is sized and shaped to receive the canister 102, which in this aspect, is cylindrical shaped. The lower portion 154 defines an open interior 156 that holds the canister 102. A vertical axis of the cylindrical-shaped canister 102 is aligned with a vertical axis of the bottle 104. The upper portion 150 is sized and shaped to position in the bottle 104 and support the canister 102. In this aspect, the upper portion 150 and the lower portion 154 may be joined by one or more ribs 158 that extend from an interior diameter 162 of the upper portion 150 to an inner opening 164 of the lower portion 154.

FIG. 2A depicts an aspect of the canister 102 inside of the cradle 112. FIG. 2B depicts canister 102 outside of the cradle 112. In one aspect, the cradle 112 may be configured to puncture the pod 106 to allow the contents of pod 106 to mix with the fluid in the bottle 104. In one aspect, the contents of the canister 102 may be delivered to the user through the outlet 108, which may go around the pod 106 and to the cap 110, where it may be taken in by the user. In another aspect, the bottle 104 may have a single outlet for both the fluid in the bottle 104 and the contents of the canister 102. In another aspect, the bottle 104 may have separate outlets for the fluid in bottle 104 and the contents of the canister 102.

The outlet 108 may pass through the circular disk 114, which may have a flow measurement device to monitor the volume and frequency of the user's intake of the contents of the canister 102.

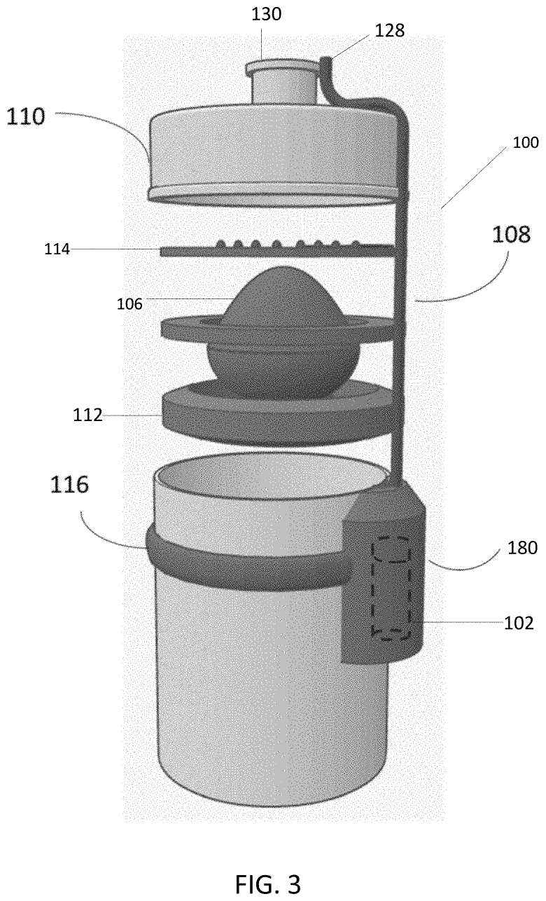

FIG. 3 illustrates an aspect with an external supplement canister 102. In this aspect, the canister 102 may be secured to the outside of the bottle 104 with a mounting bracket 116, in the form of a strap, that goes around an outer circumference of the bottle 104. The mounting bracket 116 defines an opening to receive the outer circumference of the bottle 104. In the aspect of FIG. 3, the mounting bracket 116 includes a pouch bracket 180 that holds or positions the canister 102. The mounting bracket 116 may connect with a first side of the pouch bracket 180, extend around the outer circumference of the bottle 104, and connect with a second side of the pouch bracket 180.

In other aspects, the mounting bracket 116 may be configured to pair with a specific bottle 104 shapes and sizes. The mounting bracket 116 may also be adjustable to fit a variety of bottle 104 designs.

The outlet 108 may extend along an exterior surface of the bottle 104 and the cap 110. In one aspect, outlet 108 may be configured to join with the cap 110 to allow the user to take in either the contents of the canister 102 or bottle 104 from the same outlet. In one aspect, the mounting bracket 116 may position the canister 102 so that the user may easily use separate outlets for the bottle 104 and the supplement composition from the canister 102. In one aspect, outlet 108 may be configured to capture and communicate flow data related to the canister 102 contents as part of hydration and nutritional supplement management system.

The supplement composition may include any one or more of (i) a nutritional supplement fraction, (ii) a gas fraction, (iii) an enhancer fraction, (iv) a liquid fraction and (v) a preservative fraction, wherein the enhancer fraction being a mucoadhesive to ensure adhesion of the atomized droplet to the membrane maximizing absorption. Furthermore, the particles of the composition can be positively or negatively charged so that they coalesce or disperse.

The supplement fraction may include a combination of electrolytes, vitamins, or minerals. Furthermore, the nutritional supplement matrix maybe selected from or be combination of Potassium chloride, Sodium chloride, Iron, Sodium, Calcium, Magnesium, Carbohydrates, Proteins, Zinc, Molybdenum, Caffeine, Copper, Potassium, Manganese, Chlorides, Bicarbonate and Carbonate, Aluminum, Arsenic, Bromine, Cadmium, Chromium, Sodium, Potassium, Chlorine, Cobalt, Fluorine, Iodine, Citicoline, Tyrosine Phenylalanine, Taurine, Malic Acid, Glucuronolactone, Manganese, Molybdenum Nickel, Phosphorus, Selenium, Silicon, Vanadium, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantetheine, Folic Acid, Vitamin B12, “Unofficial” B Vitamins including Choline and Inositol, Vitamin P (bioflavonoids), and flavoring agents, and/or other vital nutrients, in addition to various homeopathic/alternative substances. In one embodiment the formulation or composition of nutritional supplement matrix comprises 50 to 60 weight % of Chloride, where the chloride is from potassium chloride and sodium chloride; 30 to 40 weight % of Sodium, where the sodium is from sodium chloride; and 5 to 15 weight % of Potassium where the potassium is from potassium chloride.

The gas fraction in the above-mentioned formulation or composition comprises ambient air, oxygen, or nitrogen, wherein the gas fraction maybe in a compressed state. In one embodiment the gas fraction is comprised of oxygen wherein the concentration of oxygen is up to 95% oxygen and can mixed with other gases such as nitrogen.

The enhancer fraction in the above-mentioned formulation or composition is a mucoadhesive enhancer, an absorption enhancer, or a flavoring. Wherein the mucoadhesive enhancer fraction is selected from a group of pectin's or apple pectin's. Wherein the absorption enhancer is selected from a group of glycerin's or vegetable glycerin's. Wherein the flavoring fraction is selected from a group of natural flavoring for foods and artificial flavoring for foods.

The liquid fraction in the above-mentioned formulation or composition is selected from a group of water, distilled water, filtered water, oxygenated water or saline and where the composition is water-soluble. Wherein the amount of water is sufficient to dissolve all elements of the composition and prevent any molecules from precipitating. Furthermore, wherein the liquid fraction can be a combination of liquids and used to adjust the pH of the composition to be close to a physiological pH.

The preservative fraction in the above-mentioned formulation or composition is selected from a group of food preservatives or potassium sorbate. The food preservative fraction helps maintain and extend the shelf life of the composition.

As such, it should be understood that the disclosure is not limited to the particular aspects described herein, but that various changes and modifications may be made without departing from the spirit and scope of this novel concept as defined by the following claims. Further, many other advantages of applicant's disclosure will be apparent to those skilled in the art from the above descriptions and the claims below.

What is claimed is:

1. A beverage bottle system to dispense both a fluid and an aerosol supplement composition, comprising:
a bottle configured to hold a fluid, the bottle defining an interior;
a cap configured to engage the bottle, the cap comprising a dispensing opening;
a canister containing a supplement composition under pressure;
a cradle configured to support the canister in the system;
an outlet connecting to the canister, the outlet comprising an exterior opening, the exterior opening configured to dispense an aerosol of the supplement composition; and
the dispensing opening configured to dispense the fluid.

2. The beverage bottle system to dispense both a fluid and an aerosol supplement composition according to claim 1, wherein the exterior opening is configured to dispense the aerosol supplement composition to an oral mucosa of a user.

3. The beverage bottle system to dispense both a fluid and an aerosol supplement composition according to claim 2, wherein the dispensing opening is configured to dispense the fluid to a mouth of the user.

4. The beverage bottle system to dispense both a fluid and an aerosol supplement composition according to claim 1, wherein the canister is positioned inside of the bottle.

5. The beverage bottle system to dispense both a fluid and an aerosol supplement composition according to claim 1, wherein the outlet passes through or ends at an opening in an upper surface of the cap.

6. The beverage bottle system to dispense both a fluid and an aerosol supplement composition according to claim 1, wherein the cap rotates with respect to the bottle between locked and unlocked configurations.

7. The beverage bottle system to dispense both a fluid and an aerosol supplement composition according to claim 6, wherein, in the unlocked configuration, a user may press downward on the cap in order to dispense to the supplement composition.

8. The beverage bottle system to dispense both a fluid and an aerosol supplement composition according to claim 1, wherein the canister has a torus shape.

9. The beverage bottle system to dispense both a fluid and an aerosol supplement composition according to claim 1, further comprising a pod containing a hydration supplement.

10. The beverage bottle system to dispense both a fluid and an aerosol supplement composition according to claim 9, wherein the cradle fractures the pod to mix the hydration supplement with the fluid in the bottle.

11. The beverage bottle system to dispense both a fluid and an aerosol supplement composition according to claim 1, the cradle supports the canister, the cradle includes one or more openings to provide for fluid passage from the bottle to the dispensing opening, and the cradle is positioned on an upper rim of the bottle.

12. The beverage bottle system to dispense both a fluid and an aerosol supplement composition according to claim 1, wherein the canister is cylindrical shaped.

13. The beverage bottle system to dispense both a fluid and an aerosol supplement composition according to claim 12, the cradle includes an upper portion and a lower portion, the lower portion is sized and shaped to receive the canister.

14. The beverage bottle system to dispense both a fluid and an aerosol supplement composition according to claim 1, wherein a disk comprising a programmable logic controller is positioned over the cradle, and the programmable logic controller monitors usage statistics or make usage suggestions.

15. The beverage bottle system to dispense both a fluid and an aerosol supplement composition according to claim 1, wherein the exterior opening of the outlet is positioned within the dispensing opening of the cap.

16. The beverage bottle system to dispense both a fluid and an aerosol supplement composition according to claim 1, wherein the exterior opening of the outlet is positioned adjacent to the dispensing opening of the cap.

17. The beverage bottle system to dispense both a fluid and an aerosol supplement composition according to claim 1, wherein the exterior opening is configured to dispense the aerosol of the supplement composition separate from the fluid.

18. A beverage bottle system to dispense both a fluid and an aerosol supplement composition, comprising:
a bottle configured to hold a fluid, the bottle defining an interior;
a cap configured to engage over the interior of the bottle, the cap comprising a dispensing opening;
the dispensing opening configured to dispense the fluid;
a canister containing a supplement composition under pressure; and
an outlet connecting to the canister, the outlet comprising an exterior opening, the exterior opening configured to dispense an aerosol of the supplement composition separate from the fluid.

19. An external supplement canister for a beverage bottle, comprising:
a mounting bracket configured to extend around an outer circumference of a beverage bottle;
a canister containing a supplement composition under pressure;
the mounting bracket configured to hold the canister; and
an outlet connecting to the canister, the outlet comprising an exterior opening, the exterior opening configured to dispense an aerosol of the supplement composition.

20. The external supplement canister for a beverage bottle according to claim 19, wherein the mounting bracket includes a pouch bracket that holds or positions the canister; the mounting bracket connects with a first side of the pouch bracket, extends around the outer circumference of the beverage bottle, and connects with a second side of the pouch bracket.
